(19) United States
(12) Patent Application Publication
(10) Pub. No.: US 2011/0237970 A1
(43) Pub. Date: Sep. 29, 2011

(54) DETERMINING ELASTANCE AND RESISTANCE

(75) Inventor: Fernando Jose Isaza, Carlsbad, CA (US)

(73) Assignee: KONINKLIJKE PHILIPS ELECTRONICS N.V., EINDHOVEN (NL)

(21) Appl. No.: 13/133,194

(22) PCT Filed: Nov. 24, 2009
(86) PCT No.: PCT/IB09/55323

§ 371 Date: Jun. 7, 2011

(30) Foreign Application Priority Data
Dec. 10, 2008 (US) 61/121223

(51) Int. Cl. A61B 5/085; A61M 16/00

(52) U.S. Cl. 600/533; 128/204.23

(57) ABSTRACT

The elastance and a resistance of a subject being ventilated are determined. The determination of elastance and resistance of the breathing of the subject is made without adjusting the ventilation of the subject to facilitate the determination. That is, the determination of elastance and resistance of the subject is made without manipulating one or more parameters of the ventilation in a manner not dictated by a treatment algorithm that is designed to ventilate the subject effectively and/or comfortably.

### BACKGROUND OF THE INVENTION

1. Field of the Invention

The invention relates to determining the elastance and resistance of the breathing of a subject being ventilated.

2. Description of the Related Art

Conventional ventilation systems that mechanically ventilate patients in accordance with a treatment algorithm designed to ventilate patients efficiently and/or comfortably are known. These systems include ventilators that adjust one or more parameters of a treatment algorithm based on an elastance and/or resistance of respiration.

Conventional systems capable of determining elastance and resistance generally require extraneous adjustments to be made to, or imposed on, a ventilation treatment algorithm in order to create specific conditions within the ventilation circuit and/or the respiratory system of the patient that facilitate determination of resistance and elastance. For example, a pressure of gas in the ventilation circuit may be held static until a common pressure between the ventilation circuit and the respiratory system of the patient is reached. As another example, an extraneous pressure oscillation may be imposed on a ventilation treatment algorithm during inhalation, and the reaction of the respiratory system of the patient to this oscillation may be observed. This type of extraneous manipulation of a ventilation treatment algorithm to determine the resistance and elastance of the patient may reduce the comfort of the ventilation treatment being administered.

### SUMMARY OF THE INVENTION

One aspect of the invention relates to a system configured to determine an elastance and a resistance of the breathing of a subject. In one embodiment, the system comprises a circuit, one or more sensors, and a processor. The circuit is in communication with an airway of a subject to deliver gas to the airway of the subject and to receive gas from the airway of the subject such that the subject is mechanically ventilated by the gas delivered to and received from the airway via the circuit. The one or more sensors are configured to generate one or more output signals that convey information related to parameters of gas at or near the airway of the subject. The processor is configured to determine an elastance and a resistance of the breathing of the subject without the ventilation of the subject being adjusted to facilitate the determination, wherein the processor is configured to determine the elastance and the resistance of the breathing of the subject by (i) determining parameters of gas at or near the airway of the subject at two or more separate points in time at which muscle pressure of the subject is at or near zero based on the one or more output signals, and (ii) determining the elastance and the resistance of the breathing of the subject based on the determined parameters of the gas at or near the airway of the subject at the two or more separate points in time at which the muscle pressure of the subject is at or near zero, and wherein the elastance and the resistance of the breathing of the subject are determined by functions that describe the values of elastance and resistance as a function of the determined parameters of the gas at or near the airway of the subject at the two or more separate points in time at which the muscle pressure of the subject is at or near zero.

Another aspect of the invention relates to a method of determining an elastance and a resistance of the breathing of a subject. In one embodiment, the method comprises delivering gas to and receiving gas from an airway of a subject to mechanically ventilate the subject; generating one or more output signals that convey information related to parameters of the gas being delivered to or received from the airway of the subject; and determining an elastance and a resistance of the breathing of the subject without the ventilation of the subject being adjusted to facilitate the determination, wherein determining the elastance and the resistance of the breathing of the subject comprises: determining parameters of gas at or near the airway of the subject at two or more separate points in time at which the muscle pressure of the subject is at or near zero based on the one or more output signals, and determining the elastance and the resistance of the breathing of the subject based on the determined parameters of the gas at or near the airway of the subject at the two or more separate points in time at which the muscle pressure of the subject is at or near zero, wherein the elastance and resistance of the breathing of the subject are determined by functions that describe the values of elastance and resistance as a function of the determined parameters of the gas at or near the airway of the subject at the two or more separate points in time at which the muscle pressure of the subject is at or near zero.

Another aspect of the invention relates to a system configured to determine an elastance and a resistance of the breathing of a subject. In one embodiment, the system comprises means for delivering gas to and receiving gas from an airway of a subject to mechanically ventilate the subject; means for generating one or more output signals that convey information related to parameters of the gas being delivered to or received from the airway of the subject; and means for determining an elastance and a resistance of the breathing of the subject without the ventilation of the subject being adjusted to facilitate the determination, wherein the means for determining the elastance and the resistance of the breathing of the subject comprises: means for determining parameters of gas at or near the airway of the subject at two or more separate points in time at which the muscle pressure of the subject is at or near zero based on the one or more output signals, and means for determining the elastance and the resistance of the subject based on the determined parameters of the gas at or near the airway of the subject at the two or more separate points in time at which the muscle pressure of the subject is at or near zero, wherein the elastance and resistance of the breathing of the subject are determined by functions that describe the values of elastance and resistance as a function of the determined parameters of the gas at or near the airway of the subject at the two or more separate points in time at which the muscle pressure of the subject is at or near zero.

Another aspect of the invention relates to a system configured to determine an elastance and a resistance of the breathing of a subject. In one embodiment, the system comprises a circuit, one or more sensors, and a processor. The circuit is in communication with an airway of a subject to deliver gas to the airway of the subject and to receive gas from the airway of the subject such that the subject is mechanically ventilated by the gas delivered to and received from the airway via the circuit. The one or more sensors are configured to generate one or more output signals that convey information related to parameters of gas at or near the airway of the subject. The processor is configured to determine an elastance and a resistance of the breathing of the subject without the ventilation of the subject being adjusted to facilitate the determination, wherein the processor is configured to determine the elastance and the resistance of the breathing of the subject by (i) determining parameters of gas at or near the airway of the subject at a detection point in time at which muscle pressure of the subject and the time derivative of muscle pressure of the subject are at or near zero based on the one or more output signals, and (ii) determining the elastance and the resistance of the breathing of the subject from functions that describe the values of elastance and resistance as a function of the determined parameters of the gas at or near the airway of the subject at the detection point in time, and wherein the functions implemented to determine the values of elastance and resistance correspond to a system of equations in which elastance and resistance are unknown parameters, muscle pressure is assumed to be zero, and the time derivative of muscle pressure is assumed to be zero.

Another aspect of the invention relates to a method of determining an elastance and a resistance of the breathing of a subject. In one embodiment, the method comprises delivering gas to and receiving gas from an airway of a subject to mechanically ventilate the subject; generating one or more output signals that convey information related to parameters of the gas being delivered to or received from the airway of the subject; and determining an elastance and a resistance of the breathing of the subject without the ventilation of the subject being adjusted to facilitate the determination, wherein determining the elastance and the resistance of the breathing of the subject comprises: determining parameters of gas at or near the airway of the subject at a detection point in time at which the muscle pressure of the subject and the time derivative of muscle pressure of the subject are at or near zero based on the one or more output signals, and determining the elastance and the resistance of the breathing of the subject from functions that describe the values of elastance and resistance as a function of the determined parameters of the gas at or near the airway of the subject at the detection point in time, wherein the functions implemented to determine the values of elastance and resistance correspond to a system of equations in which elastance and resistance are unknown parameters, muscle pressure is assumed to be zero, and the time derivative of muscle pressure is assumed to be zero.

Another aspect of the invention relates to a system configured to determine an elastance and a resistance of the breathing of a subject. In one embodiment, the system comprises means for delivering gas to and receiving gas from an airway of a subject to mechanically ventilate the subject; means for generating one or more output signals that convey information related to parameters of the gas being delivered to or received from the airway of the subject; and means for determining an elastance and a resistance of the breathing of the subject without the ventilation of the subject being adjusted to facilitate the determination, wherein the means for determining the elastance and the resistance of the breathing of the subject comprises: means for determining parameters of gas at or near the airway of the subject at a detection point in time at which the muscle pressure of the subject and the time derivative of muscle pressure of the subject are at or near zero based on the one or more output signals, and means for determining the elastance and the resistance of the breathing of the subject from functions that describe the values of elastance and resistance as a function of the determined parameters of the gas at or near the airway of the subject at the detection point in time, wherein the functions implemented to determine the values of elastance and resistance correspond to a system of equations in which elastance and resistance are unknown parameters, muscle pressure is assumed to be zero, and the time derivative of muscle pressure is assumed to be zero.

Another aspect of the invention relates to a system configured to determine an elastance of the breathing of a subject. In one embodiment, the system comprises a circuit, one or more sensors, and a processor. The circuit is in communication with an airway of a subject to deliver gas to the airway of the subject and to receive gas from the airway of the subject such that the subject is mechanically ventilated by the gas delivered to and received from the airway via the circuit. The one or more sensors are configured to generate one or more output signals that convey information related to parameters of gas at or near the airway of the subject. The processor is configured to determine an elastance of the breathing of the subject without the ventilation of the subject being adjusted to facilitate the determination, wherein the processor is configured to determine the elastance of the breathing of the subject by (i) determining parameters of gas, including flow rate, at or near the airway of the subject based on the one or more output signals, (ii) identifying, from the determined parameters of gas at or near the airway of the subject, a point in time at which the subject is exhaling and the flow rate of the gas reaches an extrema, and (iii) determining the elastance of the breathing of the subject based on the parameters of gas at or near the airway of the subject at the identified point in time.

Another aspect of the invention relates to a method of determining an elastance and a resistance of the breathing of a subject. In one embodiment, the method comprises delivering gas to and receiving gas from an airway of a subject to mechanically ventilate the subject; generating one or more output signals that convey information related to parameters of the gas being delivered to or received from the airway of the subject; and determining an elastance of the breathing of the subject without the ventilation of the subject being adjusted to facilitate the determination, wherein determining the elastance of the breathing of the subject comprises: determining parameters of gas, including flow rate, at or near the airway of the subject based on the one or more output signals, identifying, from the determined parameters of gas at or near the airway of the subject, a point in time at which the subject is exhaling and the flow rate of the gas reaches an extrema, and determining the elastance of the breathing of the subject from based on the determined parameters of the gas at or near the airway of the subject at the identified point in time.

Another aspect of the invention relates to a system configured to determine an elastance and a resistance of the breathing of a subject. In one embodiment, the system comprises means for delivering gas to and receiving gas from an airway of a subject to mechanically ventilate the subject; means for generating one or more output signals that convey information related to parameters of the gas being delivered to or received from the airway of the subject; and means for determining an elastance of the breathing of the subject without the ventilation of the subject being adjusted to facilitate the determination, wherein determining the elastance of the breathing of the subject comprises: means for determining parameters of gas, including flow rate, at or near the airway of the subject based on the one or more output signals, means for identifying, from the determined parameters of gas at or near the airway of the subject, a point in time at which the subject is exhaling and the flow rate of the gas reaches an extrema, and means for determining the elastance of the breathing of the subject from based on the determined parameters of the gas at or near the airway of the subject at the identified point in time.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of “a”, “an”, and “the” include plural referents unless the context clearly dictates otherwise.

### BRIEF DESCRIPTION OF THE DRAWINGS

FIG. 1 illustrates a system configured to determine a resistance and elastance of a subject being ventilated, in accordance with one or more embodiments of the invention; and

FIG. 2 illustrates a method of determining a resistance and elastance of a subject being ventilated, according to one or more embodiments of the invention.

### DETAILED DESCRIPTION OF THE EXEMPLARY EMBODIMENTS

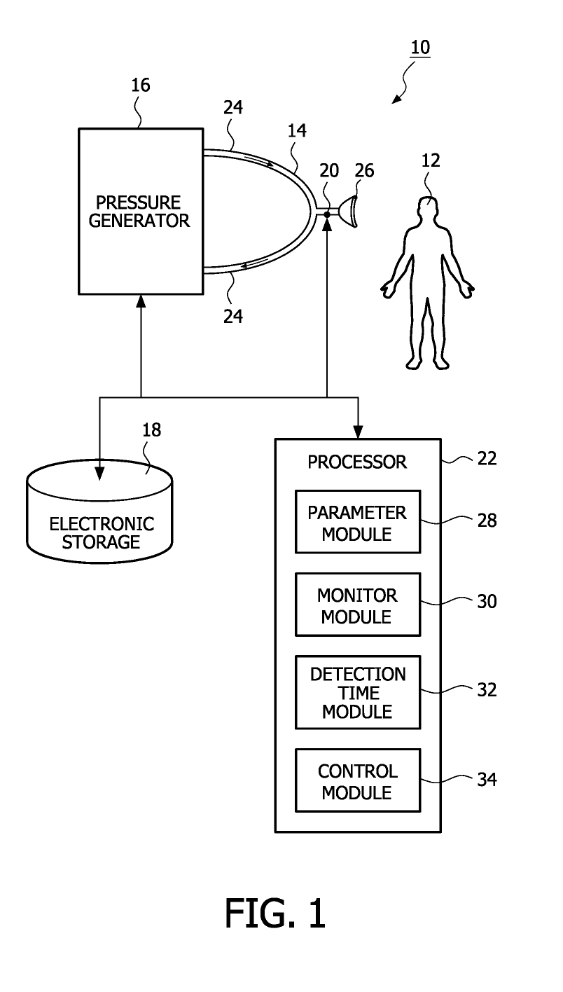

FIG. 1 illustrates a system 10 configured to determine an elastance and a resistance of the breathing of a subject 12. In particular, system 10 determines the elastance and resistance of the breathing of subject 12 as system 10 mechanically ventilates subject 12. The determination of elastance and resistance of the breathing of subject 12 is made without adjusting the ventilation of subject 12 to facilitate the determination. That is, the determination of elastance and resistance of the breathing of subject 12 is made without manipulating one or more parameters of the ventilation in a manner not dictated by a treatment algorithm that is designed to ventilate subject 12 effectively and/or comfortably. In conventional ventilation systems, manipulations of one or more ventilation parameters not dictated by a treatment algorithm are commonly made to facilitate a determination of elastance or resistance by creating a certain condition within the ventilation system (e.g., a common pressure with the lungs of subject 12, an imposed pressure oscillation on a therapeutic pressure during inhalation, etc.). From the determination of elastance and/or resistance by system 10, one or more parameters of the ventilation therapy being provided to subject 12 may be adjusted. In one embodiment, system 10 includes a gas delivery circuit 14, a pressure generator 16, electronic storage 18, sensors 20, and a processor 22.

Gas delivery circuit 14 is configured to deliver gas to and receive gas from the airway of subject 12 during ventilation. Gas delivery circuit 14 includes a conduit 24 and an interface appliance 26. Conduit 24 is a flexible conduit that runs between pressure generator 16 and interface appliance 26 to communicate gas between pressure generator 16 and interface appliance 26. Interface appliance 26 is configured to deliver gas from conduit 24 to the airway of subject 12, and to receive gas from the airway of subject 12 into conduit 24. Interface appliance 26 may include either an invasive or non-invasive appliance for communicating gas between conduit 24 and the airway of subject 12. For example, interface appliance 26 may include a nasal mask, nasal/oral mask, total face mask, endotracheal tube, or tracheal tube. Interface appliance 26 may also include a headgear assembly, such as mounting straps or a harness, for removing and fastening interface appliance 26 to subject 12. Although conduit 24 is shown as a double-limbed system, this is not intended to be limiting and conduit 24 may be formed as a single-limbed system.

Pressure generator 16 is configured to generate pressure within circuit 14 that pushes gas into and allows gas to be exhaled from the lungs of subject 12 to mechanically ventilate subject 12. It should be appreciated that although pressure generator 16 is shown in FIG. 1 and referred to in this disclosure as being a single component, pressure generator 16 may, in some embodiments, include two separate sub-systems: one that controllably provides a positive pressure to circuit 14, and one that controllably provides a pressure to circuit 14 that causes gas to be drawn out of the respiratory system of subject 12. Each of these separate sub-systems may include a source of pressure (either positive or negative), and one or more valves for controllably placing circuit 14 in communication with the source of pressure. In one embodiment, the sub-system that draws gas out of the respiratory system of subject 12 includes a valve that releases gas within conduit 24 to atmosphere. Non-limiting examples of the sources of pressure include a wall-gas source, a blower, a pressurized tank or canister of gas, atmosphere, and/or other sources of pressure. In one embodiment, pressure generator 16 also controls the composition of gas provided to subject 12 via circuit 14. For example, in this embodiment, pressure generator may control the concentration of oxygen in the gas provided to subject 12.

In one embodiment, electronic storage 18 comprises electronic storage media that electronically stores information. The electronically storage media of electronic storage 18 may include one or both of system storage that is provided integrally (i.e., substantially non-removable) with system 10 and/or removable storage that is removably connectable to system 10 via, for example, a port (e.g., a USB port, a firewire port, etc.) or a drive (e.g., a disk drive, etc.). Electronic storage 18 may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. Electronic storage 18 may store software algorithms, information determined by processor 22, information implemented in controlling system 10, information related to signals generated by sensors 20, and/or other information that enables system 10 to function properly. Electronic storage 18 may be a separate component within system 10, or electronic storage 18 may be provided integrally with one or more other components of system 10 (e.g., processor 22).

In one embodiment, sensors 20 include one or more sensors configured to monitor one or more parameters of the gas within circuit 14. As such, sensors 20 generate output signals that convey information about the one or more parameters of the gas within circuit 14. The one or more parameters may include one or more of a flow rate, a volume, a pressure, concentrations of one or more molecular species present in the gas, a temperature, a humidity, and/or other parameters. During operation, sensors 20 output one or more output signals that convey information related to the gas parameters monitored by sensors 20.

Processor 22 receives output signals generated by sensors 20 (and/or information related to output signals generated by sensors 20). Processor 22 is configured to provide information processing capabilities in system 10. As such, processor 22 may include one or more of a digital processor, an analog processor, a digital circuit designed to process information, an analog circuit designed to process information, a state machine, and/or other mechanisms for electronically processing information. Although processor 22 is shown in FIG. 1 as a single entity, this is for illustrative purposes only. In some implementations, processor 22 may include a plurality of processing units. These processing units may be physically located within the same device, or processor 22 may represent processing functionality of a plurality of devices operating in coordination.

As is shown in FIG. 1, in one embodiment, processor 22 includes a parameter module 28, a detection time module 32, a monitor module 30, a control module 34, and/or other modules. Modules 28, 30, 32, and/or 34 may be implemented in software; hardware; firmware; some combination of software, hardware, and/or firmware; and/or otherwise implemented. It should be appreciated that although modules 28, 30, 32, and/or 34 are illustrated in FIG. 1 as being co-located within a single processing unit, in implementations in which processor 22 includes multiple processing units, modules 28, 30, 32, and/or 34 may be located remotely from the other modules. Further, the description of the functionality provided by the different modules 28, 30, 32, and/or 34 described below is for illustrative purposes, and is not intended to be limiting, as any of modules 28, 30, 32, and/or 34 may provide more or less functionality than is described. For example, one or more of modules 28, 30, 32, and/or 34 may be eliminated, and some or all of its functionality may be provided by other ones of modules 28, 30, 32, and/or 34. As another example, processor 22 may include one or more additional modules that may perform some or all of the functionality attributed below to one of modules 28, 30, 32, and/or 34.

Parameter module 28 is configured to determine and/or estimate one or more parameters of gas at or near the airway of subject 12. Parameter module 28 determines and/or estimates the one or more parameters based on the one or more output signals generated by sensors 20. In one embodiment, the one or more parameters of gas at or near the airway of subject 12 comprise one or more of a flow rate of gas at or near the airway of subject 12, a pressure of gas at or near the airway of subject 12, a volume of gas within the respiratory system of subject 12 concentrations of one or more molecular species present in gas at or near the airway of subject 12, a temperature of gas at or near the airway of subject 12, a humidity of gas at or near the airway of subject 12, and/or other parameters. In one embodiment, the volume of gas within the respiratory system of subject 12 may be determined from the time integral of a measured flow rate of gas as it enters and exits the airway of subject 12. The one or more parameters determined by parameter module 28 may include time derivatives of other parameters. For example, in one embodiment, parameter module 28 is configured to determine one or more of the time derivative of the flow rate of gas at or near the airway of subject 12, the time derivative of the pressure of gas at or near the airway of subject 12, and/or other time derivatives.

Monitor module 30 monitors the elastance and the resistance of the breathing of subject 12. As such, monitor module 30 makes determinations of elastance and resistance of the breathing of subject 12 based on parameters determined by parameter module 28 at detection times determined by detection time module 32. The determination of elastance and resistance of the breathing of subject 12 by monitor module 30 does not require an adjustment to the ventilation of subject 12. In other words, the determination of elastance and resistance of the breathing of subject 12 is made without manipulating one or more parameters of the ventilation provided to subject 12 by system 10 in a manner not dictated by a treatment algorithm that is designed to ventilate subject 12 effectively and/or comfortably.

During respiration, pressure, volume, and flow rate of gas within the respiratory of subject 12 change over the course of a breathing cycle. The relation among these breathing parameters is described, in some circumstances, by the following equation:

Pₘ=Pₐ−(R·Q+E·V), (1),

where Pₘ represents muscle pressure, Pₐ represents the gas pressure at or near the airway of subject 12, R represents resistance, Q represents the flow rate of gas at or near the airway of subject 12, E represents elastance, and V represents the volume of gas in the respiratory system of subject 12. Muscle pressure is the equivalent pressure generated by the respiratory muscles to expand the thoracic cage and lungs and is a function of respiratory effort. Muscle pressure is said to be equivalent because it is not directly measurable. However, during expiration, even if the ventilation being provided by system 10 is only assisting the breathing of subject 12, muscle pressure can be assumed to be zero as subject 12 relaxes the respiratory muscles.

In equation (1), there are three parameters that are typically not measured directly by conventional ventilators. These three parameters are muscle pressure, resistance, and elastance. If muscle pressure can be assigned a value (e.g., through estimation or assumption), then equation (1) can be considered to have only two unknowns.

In one embodiment, monitor module 30 determines elastance and resistance according to equation (1) based on (i) the gas pressure at or near the airway of subject 12, (ii) the flow rate of gas into or out of the airway of subject 12, and (iii) the volume of gas in the respiratory system of subject 12. For example, from measurements of these parameters at two separate points in time when muscle pressure can be assumed to be zero, equation (1) can be used to generate a set of equations that can be solved for resistance and elastance. As such, the measurements of these parameters at two points in time where muscle pressure is assumed to be at or near zero can be implemented by monitor module 30 to determine the resistance and elastance of the breathing of subject 12 for those two points in time.

Muscle pressure can be assumed to be zero at points in time where it is likely that subject 12 is not exerting any effort to breath. For example, in ventilated patients, exhalation is typically a relaxation of the respiratory muscles. As such, during exhalation muscle pressure may assumed to be zero. As another example, if a patient is not capable of exerting any effort in breathing, muscle pressure may assumed to be zero during inhalation as well as exhalation. Patients incapable of exerting effort in breathing include patients who have been over-supported, patients with extreme and/or degenerative damage to their respiratory systems and/or brain function, patients paralyzed by drugs, and/or other patients.

By way of non-limiting example, if muscle pressure is assumed to be zero at two detection times t₁ and t₂, equation (1) yields the following relationships:

0=Pₐ₁−(R·Q₁+E·V₁), (2)

and

0=Pₐ₂−(R·Q₂+E·V₂), (3)

where Pₐ₁ and Pₐ₂ represent gas pressure at or near the airway of subject 12 at detection times t₁ and t₂, respectively, Q₁ and Q₂ represent the flow rate of gas at or near the airway of subject 12 at detection times t₁ and t₂, respectively, and V₁ and V₂ represent the volume of gas in the respiratory system of subject 12 at detection times t₁ and t₂, respectively. The system of equations (2) and (3) then yield the following solutions for resistance and elastance, which in one embodiment are implemented to determine the resistance and elastance of the breathing of subject 12 by monitor module 30:

$$\begin{matrix}R = \frac{{V}_{1} \cdot {P}_{a 2} - {V}_{2} \cdot {P}_{a 1}}{{Q}_{2} \cdot {V}_{1} - {Q}_{1} \cdot {V}_{2}} , \\ and & ( 4 ) \\ E = \frac{{Q}_{1} \cdot {P}_{a 2} - {Q}_{2} \cdot {P}_{a 1}}{{Q}_{1} \cdot {V}_{2} - {Q}_{2} \cdot {V}_{1}} . & ( 5 )\end{matrix}$$

As another non-limiting example, according to equation (1), estimates or guesses for resistance and elastance, in conjunction with measured values for Pₐ₁, Q₁, and V₁ will yield an estimate for muscle pressure at t₁ as:

Pₘ₁ₑ=(Rₑ·Q₁+Eₑ·V₁)−Pₐ₁, (6)

where Pₘ₁ₑ represents the estimate of Pₘ at t₁, Rₑ represents the estimate for resistance, and Eₑ represents the estimate for elastance. Similarly, the estimates for resistance and elastance, in conjunction with measured values for Pₐ₂, Q₂, and V₂, will yield an estimate for muscle pressure at t₂ as:

Pₘ₂ₑ=(Rₑ·Q₂+Eₑ·V₂)−Pₐ₂, (7)

where Pₘ₂ₑ represents the estimate of Pₘ at t₂.

By substituting the measured values for Pₐ₁, Q₁, and V₁ into equation (1), and then subtracting the resulting equation from equation (6), the following relationship is derived:

Pₘ₁ₑ−Pₘ₁=(R−Rₑ)·Q₁+(E−Eₑ)·V₁. (8)

If we define the difference between the estimated muscle pressure for t₁ and the actual muscle pressure at t₁(i.e., Pₘ₁ₑ−Pₘ₁) as ΔPₘ₁, the difference between the estimated resistance and the actual resistance (i.e., R−Rₑ) as ΔR, the difference between the estimated elastance and the actual elastance (i.e., E−Eₑ) as ΔE, then equation (8) can be rewritten as:

ΔPₘ₁=ΔR·Q₁+ΔE·V₁. (9)

Similar steps with respect to equation (7), rather than equation (6), yield:

ΔPₘ₂=ΔR·Q₂+ΔE·V₂ (10).

If we use equations (9) and (10) as a system of equations with two unknowns (ΔR and ΔE), we can solve for ΔR and ΔE as follows:

$$\begin{matrix}Δ R = \frac{Δ {P}_{m 1} - ( \frac{Δ {P}_{m 1} \cdot {Q}_{1} - Δ {P}_{m 1} \cdot {Q}_{2}}{{Q}_{1} \cdot {V}_{2} - {Q}_{2} \cdot {V}_{1}} ) \cdot {V}_{1}}{{Q}_{1}} , \\ and & ( 11 ) \\ Δ E = \frac{Δ {P}_{m 2} \cdot {Q}_{1} - Δ {P}_{m 1} \cdot {Q}_{2}}{{Q}_{1} \cdot {V}_{2} - {Q}_{2} \cdot {V}_{1}} . & ( 12 )\end{matrix}$$

As was discussed above, if both t₁ and t₂ occur during exhalation by subject 12, then Pₘ₁ and Pₘ₂ can be assumed to be zero, and ΔPₘ₁ and ΔPₘ₂ go to Pₘ₁ₑ and Pₘ₂ₑ, respectively. If Pₘ₁ₑ and Pₘ₂ₑ are substituted for ΔPₘ₁ and ΔPₘ₂, respectively, then equations (11) and (12) can be rewritten and solved for E and R as:

$$\begin{matrix} R = {R}_{e} + [ \frac{ {P}_{m 1 e} - ( \frac{{P}_{m 1 e} \cdot {Q}_{1} - {P}_{m 1 e} \cdot {Q}_{2}}{{Q}_{1} \cdot {V}_{2} - {Q}_{2} \cdot {V}_{1}} ) \cdot {V}_{1}}{{Q}_{1}} ] , \\ and & ( 13 ) \\ E = {E}_{e} + [ \frac{{P}_{m 2 e} \cdot {Q}_{1} - {P}_{m 1 e} \cdot {Q}_{2}}{{Q}_{1} \cdot {V}_{2} - {Q}_{2} \cdot {V}_{1}} ] . & ( 14 )\end{matrix}$$

In one embodiment, monitor module 30 implements equations (13) and (14) to determine the elastance and resistance of subject 12, using previous determinations of elastance and resistance to determine Pₘ₁ₑ and Pₘ₂ₑ, and as Eₑ and Rₑ. Equations (13) and (14) will even provide accurate initial calculations of elastance and resistance (e.g., prior to there being previous determinations of elastance and resistance) using any reasonable estimations for estimated elastance and resistance. For example, any value within several orders of magnitude (e.g., not approaching infinity) of the actual values of elastance and resistance will yield accurate determinations of elastance and resistance.

As will be appreciated, during periods of time where muscle pressure remains at or near zero (e.g., during exhalation, etc.), muscle pressure is constant over time. As such, in one embodiment, rather than determining elastance and resistance according to functions that calculate elastance and resistance as a function of values of parameters of gas at or near the airway of subject 12 at two or more separate points in time, monitor module 30 determines elastance and resistance according to functions that calculate elastance and resistance as a function of parameters of gas at or near the airway of subject 12 at a single point in time. These functions can be derived from a system of equations that expresses muscle pressure as a function of parameters of gas at or near the airway of subject 12 (e.g., equation (1)) and the time derivative of this equation.

By way of non-limiting example, the time derivative of equation (1) can be expressed as follows:

$$\frac{d {P}_{m}}{d t} = ( R \cdot \frac{d Q}{d t} + E \cdot \frac{d V}{d t} ) - \frac{d {P}_{a}}{d t} . \qquad ( 15 )$$

During periods of time where muscle pressure remains at or near zero (e.g., during exhalation, etc.), muscle pressure is constant over time, thus its time derivative is zero (0). Further, the time derivative of volume is flow. As such, equation (15) can be expressed during periods of time in which muscle pressure is assumed to remain constant at or near zero as:

$$0 = ( R \cdot \frac{d Q}{d t} + E \cdot Q ) - \frac{d {P}_{a}}{d t} . \qquad ( 16 )$$

The time derivatives of flow and Pₐ at a specific point in time are parameters of the gas that can be determined from measurements of flow and Pa over time. In one embodiment, these parameters are determined by parameter module 28. Thus, equation (16) includes only two unknowns, resistance and elastance, and equation (16) can be used along with equation (1) to form a system of two equations with two common unknowns. This system of equations can be solved for resistance and elastance at detection time t₁ as follows:

$$\begin{matrix}R = \frac{\frac{{Q}_{1} {P}_{a 1}}{{V}_{1}} - {\dot{P}}_{a 1}}{\frac{1}{{V}_{1}} - {\dot{Q}}_{1}} \\ and & ( 17 ) \\ e = \frac{{\dot{P}}_{a 1} - {\dot{Q}}_{1} [ \frac{\frac{{Q}_{1} {P}_{a 1}}{{V}_{1}} - {\dot{P}}_{a 1}}{\frac{1}{{V}_{1}} - {\dot{Q}}_{1}} ]}{{Q}_{1}} , & ( 18 )\end{matrix}$$

where \dot{P}ₐ₁ represents the time derivative of Pₐ evaluated at detection time t₁, and \dot{Q}₁ represents the time derivative of flow evaluated at detection time t₁. In one embodiment, monitor module 30 implements equations (17) and (18) to determine the elastance and resistance of subject 12.

During exhalation, the flow rate of gas at or near the airway of subject 12 reaches an extrema at the point in time where the flow rate of the gas leaving the airway of subject 12 reaches its maximum value. At this point in time, the time derivative of the flow is zero. Accordingly, at this point in time equation (16) can be rewritten as:

$$E = \frac{( \frac{d {P}_{a}}{d t} )}{Q} . \qquad ( 19 )$$

In one embodiment, monitor module 30 implements equation (19) at a detection time determined to correspond to a point in time during an exhalation by subject 12 at which the flow rate of gas at or near the airway of subject 12 reaches an extrema in order to calculate elastance. Monitor module 30 then implements this calculation of elastance to determine the resistance of subject 12. For example, at a second detection time (at which muscle pressure can be assumed to be zero) monitor module 30 implements the determination of elastance made via equation (19) according to the following function, which is derived from equation (1):

R=Pₐ−E·V. (20)

Detection time module 32 is configured to determine one or more detection times at which determinations of parameters by parameter module 28 should be implemented to determine the resistance and elastance of the lungs of subject 12 by monitor module 30. In one embodiment, the detection times occur during points in time at which muscle pressure is assumed to be zero in order to facilitate determination of elastance and resistance according to, for example, one of the techniques described above. In one embodiment, the detection times include one detection time that occurs during exhalation at or near an extrema in the flow rate of gas at or near the airway of subject 12.

Detection time module 32 may detect the occurrence of one or more suitable detection times based on one or more of the controls of pressure generator 16, the parameters determined by parameter module 28, and/or otherwise determined. In one embodiment, a first and second detection time for a determination of elastance and resistance may be determined during a common breathing cycle (e.g., during same exhalation, during the same inhalation, during the inhalation and exhalation of the same breath). In one embodiment, a first and second detection time for a determination of elastance and resistance may be determined during separate breathing cycles (e.g., during separate exhalations or inhalations, or during the inhalation and the exhalation phases of different breaths). The detection times may be determined by detection time module 32 to enhance an accuracy and/or a precision of determinations of elastance and resistance. For example, in an embodiment in which elastance and resistance are determined from parameters of the gas at or near the airway of subject 12 at two separate points in time, the first detection time may be determined to be relatively close to the beginning of the exhalation of the common breathing cycle and the second detection time may be determined to be relatively close to the end of the exhalation of the common breathing cycle.

Control module 34 is configured to control the operation of pressure generator 24 in ventilating subject 12. In one embodiment, based on a determination of elastance and/or resistance by monitor module 30, control module 24 may adjust one or more parameters of the ventilation of subject 12. For example, the one or more parameters of the ventilation of subject 12 that are adjusted may include one or more of work of breathing factor, adjustable rise setting, inspiratory time setting, pressure target setting, PEEP setting, trigger sensitivity setting, cycle sensitivity setting, peak flow setting, tidal volume setting, and/or other parameters.

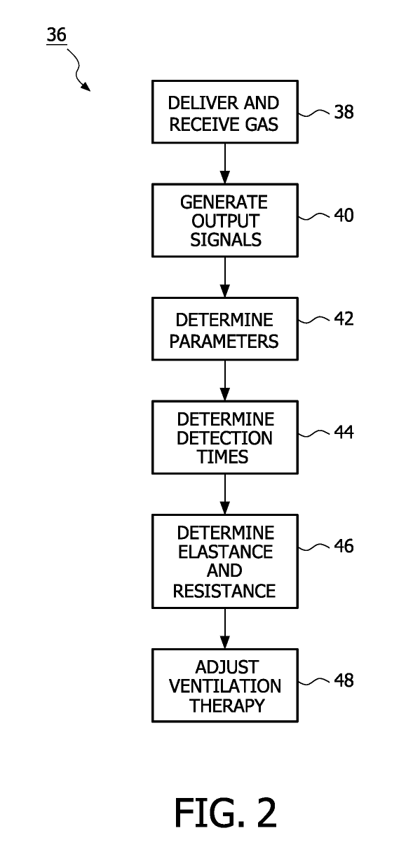

FIG. 2 illustrates a method 36 of determining an elastance and a resistance of the breathing of a subject. The operations of method 36 presented below are intended to be illustrative. In some embodiments, method 36 may be accomplished with one or more additional operations not described, and/or without one or more of the operations discussed. Additionally, the order in which the operations of method 36 are illustrated in FIG. 2 and described below is not intended to be limiting. Further, although method 36 is described in the context of system 10 (shown in FIG. 1 and described above), method 36 may be implemented in a variety of contexts without departing from the scope of this disclosure.

At an operation 38, gas is delivered to and received from an airway of a subject to ventilate the subject. In one embodiment, operation 38 may be performed by a pressure generator and circuit that are the same as or similar to pressure generator 16 and circuit 14 (shown in FIG. 1 and described above).

At an operation 40, one or more output signals are generated that convey information related to parameters of the gas being delivered to or received from the airway of the subject. In one embodiment, operation 40 is performed by one or more sensors that are the same as or similar to sensors 20 (shown in FIG. 1 and described above).

At an operation 42, one or more parameters of the gas being delivered to or received from the airway of the subject are determined from the output signals generated at operation 40. In one embodiment, operation 42 is performed by a parameter module that is the same as or similar to parameter module 28 (shown in FIG. 1 and described above).

At an operation 44, one or more detection times are determined Detection times are times during which the parameters determined at operation 42 will enable a determination of elastance and resistance. For example, the detection times may include points in time at which the muscle pressure of the subject is at or near zero, points in time during exhalation at which the flow rate of gas at or near the airway of subject 12 reaches an extrema, and/or other points in time. In one embodiment, operation 44 is performed by a detection time module that is the same as or similar to detection time module 32 (shown in FIG. 1 and described above).

At an operation 46, elastance and resistance of the breathing of the subject are determined. The determination of elastance and resistance at operation 46 is based on gas parameters determined at operation 42 for detection times determined at operation 44. The determination of elastance and resistance is not facilitated by a manipulation of the ventilation provided to the subject via operation 38. In one embodiment, operation 46 is performed by a monitor module that is the same as or similar to monitor module 30 (shown in FIG. 1 and described above).

At an operation 48, one or more parameters of the ventilation being provided to the subject via operation 38 are adjusted based on the determination of elastance and/or resistance at operation 46. In one embodiment, operation 48 is performed by a control module that is the same as or similar to control module 34 (shown in FIG. 1 and described above).

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

What is claimed is:

1. A system configured to determine an elastance and a resistance of the breathing of a subject, the system comprising:
a circuit in communication with an airway of a subject to deliver gas to the airway of the subject and to receive gas from the airway such that the subject is mechanically ventilated by the gas delivered to and received from the airway via the circuit;
a sensor configured to generate a output signal that conveys information related to a parameter of gas at or near the airway; and
a processor configured to determine an elastance and a resistance of the subject without the ventilation of the subject being adjusted to facilitate the determination, wherein the processor is configured to determine the elastance and the resistance of the breathing of the subject by (i) determining parameters of gas at or near the airway of the subject at two or more separate points in time at which muscle pressure of the subject is at or near zero based on the output signal, and (ii) determining the elastance and the resistance of the breathing of the subject based on the determined parameters of the gas at or near the airway of the subject at the two or more separate points in time at which the muscle pressure of the subject is at or near zero, and wherein the elastance and the resistance are determined by functions that describe the values of elastance and resistance as a function of the determined parameters of the gas at or near the airway at the two or more separate points in time at which the muscle pressure of the subject is at or near zero.

2. The system of claim 1, wherein the functions implemented to determine the elastance and the resistance as a function the determined parameters of the gas at or near the airway at the two or more separate points in time at which the muscle pressure is at or near zero correspond to a system of equations in which elastance and resistance are unknown parameters and muscle pressure is assumed to be zero.

3. The system of claim 1, wherein the parameters of gas determined by the processor at the two or more separate points in time at which the muscle pressure of the subject is at or near zero comprise a flow rate of gas at or near the airway, a pressure of gas at or near the airway of the subject, and a volume of gas within the respiratory system of the subject.

4. The system of claim 1, wherein the two or more separate points in time at which the muscle pressure of the subject is at or near zero include at least two separate points in time that occur during the same breath.

5. The system of claim 1, wherein the elastance and the resistance of the breathing of the subject is determined by the processor from the determined parameters of the gas at or near the airway at the two or more separate points in time at which the muscle pressure of the subject is at or near zero according to the equation of motion for the respiratory system.

6. The system of claim 1, wherein the processor is further configured to adjust one more parameters of the ventilation provided to the subject via the circuit in accordance with the determined elastance and/or resistance.

7. A method of determining an elastance and a resistance of the breathing of a subject, the method comprising:
(a) delivering gas to and receiving gas from an airway of a subject to mechanically ventilate the subject;
(b) generating an output signal that conveys information related to a parameter of the gas being delivered to or received from the airway of the subject; and
(c) determining an elastance and a resistance of the subject without the ventilation of the subject being adjusted to facilitate the determination, wherein determining the elastance and the resistance comprises:
(1) determining parameters of gas at or near the airway at two or more separate points in time at which the muscle pressure of the subject is at or near zero based on the output signal, and
(2) determining the elastance and the resistance based on the determined parameters of the gas at or near the airway at the two or more separate points in time at which the muscle pressure of the subject is at or near zero, and wherein the elastance and resistance are determined by functions that describe the values of elastance and resistance as a function of the determined parameters of the gas at or near the airway at the two or more separate points in time at which the muscle pressure of the subject is at or near zero.

8. The method of claim 7, wherein the functions implemented to determine the elastance and the resistance as a function of the determined parameters of the gas at or near the airway of the subject at the two or more separate points in time at which the muscle pressure of the subject is at or near zero correspond to a system of equations in which elastance and resistance are unknown parameters and muscle pressure is assumed to be zero.

9. The method of claim 7, wherein the parameters of gas determined at the two or more or more separate points in time at which the muscle pressure of the subject is at or near zero comprise a flow of gas at or near the airway of the subject, a pressure of gas at or near the airway of the subject, and a volume of gas within the respiratory system of the subject.

10. The method of claim 7, wherein the two or more separate points in time at which the muscle pressure of the subject is at or near zero include at least two separate points in time that occur during the same breath.

11. The method of claim 7, wherein the elastance and the resistance is determined from the determined parameters of the gas at or near the airway of the subject at the two or more separate points in time at which the muscle pressure of the subject is at or near zero according to the equation of motion for the respiratory system.

12. The method of claim 7, further comprising adjusting one more parameters of the delivery of gas to and/or reception of gas from the airway of the subject in accordance with the determined elastance and/or resistance.

13. A system configured to determine an elastance and a resistance of the breathing of a subject, the system comprising:
(a) means for delivering gas to and receiving gas from an airway of a subject to mechanically ventilate the subject;
(b) means for generating an output signal that conveys information related to a parameter of the gas being delivered to or received from the airway of the subject; and
(c) means for determining an elastance and a resistance of the subject without the ventilation of the subject being adjusted to facilitate the determination, wherein the means for determining the elastance and the resistance comprises:
(1) means for determining parameters of gas at or near the airway of the subject at two or more separate points in time at which the muscle pressure of the subject is at or near zero based on the output signal, and
(2) means for determining the elastance and the resistance of the subject based on the determined parameters of the gas at or near the airway of the subject at the two or more separate points in time at which the muscle pressure of the subject is at or near zero, and wherein the elastance and resistance of the breathing of the subject are determined by functions that describe the values of elastance and resistance as a function of the determined parameters of the gas at or near the airway of the subject at the two or more separate points in time at which the muscle pressure of the subject is at or near zero.

14. The system of claim 13, wherein the functions implemented to determine the elastance and the resistance based on the determined parameters of the gas at or near the airway of the subject at the two or more separate points in time at which the muscle pressure of the subject is at or near zero correspond to a system of equations in which elastance and resistance are unknown parameters and muscle pressure is assumed to be zero.

15. The system of claim 13, wherein the parameters of gas determined at the two or more separate points in time at which the muscle pressure of the subject is at or near zero comprise a flow of gas at or near the airway of the subject, a pressure of gas at or near the airway of the subject, and a volume of gas within the respiratory system of the subject.

16. The system of claim 13, wherein the two or more separate points in time at which the muscle pressure of the subject is at or near zero include at least two separate points in time that occur during the same breath.

17. The system of claim 13, wherein the elastance and the resistance of is determined from the determined parameters of the gas at or near the airway of the subject at the two or more separate points in time at which the muscle pressure of the subject is at or near zero according to the equation of motion for the respiratory system.

18. The system of claim 13, further comprising means for adjusting one more parameters of the delivery of gas to and/or reception of gas from the airway of the subject in accordance with the determined elastance and/or resistance.

19. A system configured to determine an elastance and a resistance of the breathing of a subject, the system comprising:
a circuit in communication with an airway of a subject to deliver gas to the airway of the subject and to receive gas from the airway of the subject such that the subject is mechanically ventilated by the gas delivered to and received from the airway via the circuit;
a sensor configured to generate an output signal that conveys information related to a parameter of gas at or near the airway of the subject; and
a processor configured to determine an elastance and a resistance of the breathing of the subject without the ventilation of the subject being adjusted to facilitate the determination, wherein the processor is configured to determine the elastance and the resistance of the breathing of the subject by (i) determining parameters of gas at or near the airway of the subject at a detection point in time at which muscle pressure of the subject and the time derivative of muscle pressure of the subject are at or near zero based on the output signal, and (ii) determining the elastance and the resistance of the subject from functions that describe the values of elastance and resistance as a function of the determined parameters of the gas at or near the airway at the detection point in time, and wherein the functions implemented to determine the values of elastance and resistance correspond to a system of equations in which elastance and resistance are unknown parameters, muscle pressure is assumed to be zero, and the time derivative of muscle pressure is assumed to be zero.

20. The system of claim 19, wherein the detection point in time occurs during exhalation.

21. The system of claim 19, wherein the parameters of the gas at or near the airway of the subject comprise the time derivative of the flow rate of gas at or near the airway of the subject and the time derivative of the pressure of gas at or near the airway of the subject.

22. The system of claim 19, wherein the system of equations includes an equation that describes the muscle pressure of the subject as a function of the parameters of gas at or near the airway of the subject and the time derivative of this equation.

23. The system of claim 22, wherein the system of equations are derived from the equation of motion for the respiratory system and the time derivative of the equation of motion.

24. The system of claim 19, wherein the processor is further configured to adjust one or more parameters of the ventilation provided to the subject via the circuit in accordance with the determined elastance and/or resistance.

25. A method of determining an elastance and a resistance of the breathing of a subject, the method comprising:
(a) delivering gas to and receiving gas from an airway of a subject to mechanically ventilate the subject;
(b) generating an output signal that conveys information related to a parameter of the gas being delivered to or received from the airway; and
(c) determining an elastance and a resistance of the subject without the ventilation of the subject being adjusted to facilitate the determination, wherein determining the elastance and the resistance of the breathing of the subject comprises:
(1) determining parameters of gas at or near the airway at a detection point in time at which the muscle pressure of the subject and the time derivative of muscle pressure of the subject are at or near zero based on the output signal, and
(2) determining the elastance and the resistance from functions that describe the values of elastance and resistance as a function of the determined parameters of the gas at or near the airway of the subject at the detection point in time, and wherein the functions implemented to determine the values of elastance and resistance correspond to a system of equations in which elastance and resistance are unknown parameters, muscle pressure is assumed to be zero, and the time derivative of muscle pressure is assumed to be zero.

26. The method of claim 25, wherein the detection point in time occurs during exhalation.

27. The method of claim 25, wherein the parameters of the gas at or near the airway of the subject comprise the time derivative of the flow rate of gas at or near the airway of the subject and the time derivative of the pressure of gas at or near the airway of the subject.

28. The method of claim 25, wherein the system of equations includes an equation that describes the muscle pressure of the subject as a function of the parameters of gas at or near the airway of the subject and the time derivative of this equation.

29. The method of claim 28, wherein the system of equations are derived from the equation of motion for the respiratory system and the time derivative of the equation of motion.

30. The method of claim 25, further comprising adjusting one or more parameters of the ventilation provided to the subject in accordance with the determined elastance and/or resistance.

31. A system configured to determine an elastance and a resistance of the breathing of a subject, the system comprising:
(a) means for delivering gas to and receiving gas from an airway of a subject to mechanically ventilate the subject;
(b) means for generating an output signal that conveys information related to a parameter of the gas being delivered to or received from the airway of the subject; and
(c) means for determining an elastance and a resistance of the subject without the ventilation of the subject being adjusted to facilitate the determination, wherein the means for determining the elastance and the resistance of the breathing of the subject comprises:
(1) means for determining parameters of gas at or near the airway at a detection point in time at which the muscle pressure of the subject and the time derivative of muscle pressure of the subject are at or near zero based on the output signal, and
(2) means for determining the elastance and the resistance from functions that describe the values of elastance and resistance as a function of the determined parameters of the gas at or near the airway of the subject at the detection point in time, and wherein the functions implemented to determine the values of elastance and resistance correspond to a system of equations in which elastance and resistance are unknown parameters, muscle pressure is assumed to be zero, and the time derivative of muscle pressure is assumed to be zero.

32. The system of claim 31, wherein the detection point in time occurs during exhalation.

33. The system of claim 31, wherein the parameters of the gas at or near the airway of the subject comprise the time derivative of the flow rate of the gas at or near the airway of the subject and the time derivative of the pressure of the gas at or near the airway of the subject.

34. The system of claim 31, wherein the system of equations includes an equation that describes the muscle pressure of the subject as a function of the parameters of the gas at or near the airway of the subject and the time derivative of this equation.

35. The system of claim 34, wherein the system of equations are derived from the equation of motion for the respiratory system and the time derivative of the equation of motion.

36. The system of claim 31, further comprising means for adjusting one or more parameters of the ventilation provided to the subject in accordance with the determined elastance and/or resistance.

37. A system configured to determine an elastance of the breathing of a subject, the system comprising:
a circuit in communication with an airway of a subject to deliver gas to the airway of the subject and to receive gas from the airway of the subject such that the subject is mechanically ventilated by the gas delivered to and received from the airway via the circuit;
a sensor configured to generate an output signal that conveys information related to a parameter of gas at or near the airway of the subject; and
a processor configured to determine an elastance of the breathing of the subject without the ventilation of the subject being adjusted to facilitate the determination, wherein the processor is configured to determine the elastance of the breathing of the subject by (i) determining parameters of gas, including flow rate, at or near the airway of the subject based on the output signal, (ii) identifying, from the determined parameters of gas at or near the airway of the subject, a point in time at which the subject is exhaling and the flow rate of the gas reaches an extrema, and (iii) determining the elastance of the breathing of the subject based on the parameters of gas at or near the airway of the subject at the identified point in time.

38. The system of claim 37, wherein the processor is further configured to determine the resistance of the breathing of the subject based on the determined elastance and the parameters of the gas at or near the airway of the subject at a second point in time.

39. The system of claim 38, wherein the muscle pressure of the subject at the second point in time is at or near zero.

40. The system of claim 37, wherein the processor is further configured to adjust one or more parameters of the ventilation provided to the subject via the circuit in accordance with the determined elastance.

41. A method of determining an elastance and a resistance of the breathing of a subject, the method comprising:
(a) delivering gas to and receiving gas from an airway of a subject to mechanically ventilate the subject;
(b) generating an output signal that conveys information related to a parameter of the gas being delivered to or received from the airway of the subject; and
(c) determining an elastance of the subject without the ventilation of the subject being adjusted to facilitate the determination, wherein determining the elastance of the breathing of the subject comprises:
(1) determining parameters of gas, including flow rate, at or near the airway of the subject based on the output signal,
(2) identifying, from the determined parameters of gas at or near the airway of the subject, a point in time at which the subject is exhaling and the flow rate of the gas reaches an extrema, and
(d) determining the elastance of the subject from based on the determined parameters of the gas at or near the airway of the subject at the identified point in time.

42. The method of claim 41, further comprising determining the resistance of the breathing of the subject based on the determined elastance and the parameters of the gas at or near the airway of the subject at a second point in time.

43. The method of claim 42, wherein the muscle pressure of the subject at the second point in time is at or near zero.

44. The method of claim 41, further comprising adjusting one or more parameters of the ventilation provided to the subject in accordance with the determined elastance.

45. A system configured to determine an elastance and a resistance of the breathing of a subject, the system comprising:
(a) means for delivering gas to and receiving gas from an airway of a subject to mechanically ventilate the subject;
(b) means for generating an output signal that conveys information related to a parameter of the gas being delivered to or received from the airway of the subject; and
(c) means for determining an elastance of the subject without the ventilation of the subject being adjusted to facilitate the determination, wherein determining the elastance of the breathing of the subject comprises:
(1) means for determining parameters of gas, including flow rate, at or near the airway of the subject based on the output signal,
(2) means for identifying, from the determined parameters of gas at or near the airway of the subject, a point in time at which the subject is exhaling and the flow rate of the gas reaches an extrema, and
(3) means for determining the elastance of the breathing of the subject from based on the determined parameters of the gas at or near the airway of the subject at the identified point in time.

46. The method of claim 45, further comprising means for determining the resistance based on the determined elastance and the parameters of the gas at or near the airway of the subject at a second point in time.

47. The method of claim 46, wherein the muscle pressure of the subject at the second point in time is at or near zero.

48. The method of claim 45, further means for comprising adjusting one or more parameters of the ventilation provided to the subject in accordance with the determined elastance.
